(19) United States
(12) Patent Application Publication
(10) Pub. No.: US 2026/0263483 A1
(43) Pub. Date: Sep. 10, 2026

(54) LOW DOSE THERAPEUTIC SUPPLEMENT TO MODULATE CYTOKINES

(71) Applicants: VIRBAC CORPORATION, Westlake, TX (US); FLORIDA RESEARCH GROUP LLC, Tampa, FL (US)

(72) Inventor: Kotha Sekharam, Tampa, FL (US)

(73) Assignees: VIRBAC CORPORATION, Westlake, TX (US); FLORIDA RESEARCH GROUP LLC, Tampa, FL (US)

(21) Appl. No.: 19/160,850

(22) PCT Filed: Mar. 1, 2024
(86) PCT No.: PCT/US2024/017985

§ 371 Date: Aug. 28, 2025

Related U.S. Application Data
(60) Provisional application No. 63/487,873, filed on Mar. 1, 2023.
(60) Provisional application No. 63/487,887, filed on Mar. 2, 2023.
(60) Provisional application No. 63/492,546, filed on Mar. 28, 2023.
(60) Provisional application No. 63/492,549, filed on Mar. 28, 2023.

(51) Int. Cl. A61K 31/56; A61K 9/16

(52) U.S. Cl. CPC A61K 31/56; A61K 9/1635; A61K 9/1694

(57) ABSTRACT

The present invention provides a new and improved method to prepare a stable amorphous pentacyclic triterpenoid, a composition prepared with such pentacyclic triterpenoid, and a method to mitigate effects caused by pro-inflammatory cytokines in mammals, including humans, by providing an effective dose of a pentacyclic triterpenoid that has been processed according to the present invention. The present invention also provides an effective low-dose therapeutic composition comprising a pentacyclic triterpenoid that has been processed according to the present invention.

### CROSS-REFERENCE TO RELATED APPLICATION

This application claims priority to U.S. Provisional Patent Application Ser. Nos. 63/487,873, filed Mar. 1, 2023; 63/487,887, filed Mar. 2, 2023; 63/492,546, filed Mar. 28, 2023; and 63/492,549, filed Mar. 28, 2023; all of which are incorporated herein by reference in their entirety.

### BACKGROUND OF THE INVENTION

Cytokines are a diverse group of small, secreted proteins released for the purpose of intercellular signaling and communication. Cytokines are produced in response to invading pathogens to stimulate, recruit, and proliferate immune cells. Cytokines may act on the cells that secrete them, on nearby cells, or in some instances on distant cells.

Proinflammatory cytokines are produced predominantly by activated macrophages and are involved in the up-regulation of inflammatory reactions. Key pro-inflammatory cytokines include interleukin 1 (IL-1), interleukin 6 (IL-6), and tumor necrosis factor alpha (TNF-α). They are crucial for coordinating cell-mediated immune response and play a critical role in modulating the immune system. Pro-inflammatory cytokines generally regulate growth, cell activation, differentiation, and homing of the immune cells to the sites of infection with the aim to control and eradicate the intracellular pathogens, including viruses.

Excessive or uncontrolled release of pro-inflammatory cytokines in the body causes ‘cytokine storm.’ Cytokine storms are associated with a wide variety of infectious and noninfectious diseases. Inflammation associated with a cytokine storm begins at a local site and spreads throughout the body via the systemic circulation. During viral infections, such as severe acute respiratory syndrome coronavirus 2 (SARS-CoV-2), respiratory syncytial virus (RSV), Parvovirus B19, and Influenza viruses, the level of the pro-inflammatory cytokines is elevated and decreased upon clearance of the virus.

COVID-19 patients were found to have an elevated level of interleukin 1β (IL-1β), IL-6, interleukin 10 (IL-10), interferon gamma (IFN-γ), TNF-α, interferon gamma-induced protein (IP10), granulocyte colony-stimulating factor (GCSF), and monocyte chemoattractant protein-1 (MCP1). In addition, ICU-admitted patients have shown higher levels of plasma cytokines compared to their non-ICU-admitted counterparts, indicating that the severity of COVID-19 illness is amplified by cytokine levels. See C. Huang, Y. et al., Clinical Features of Patients Infected with 2019 Novel Coronavirus in Wuhan, China, Lancet, 395, 497-506 (2020); P. Mehta et al., Covid-19: Consider Cytokine Storm Syndromes and Immunosuppression, Lancet, 395, 1033-1034 (2020); B. Diao, et al., Reduction and Functional Exhaustion of T Cells in Patients with Coronavirus Disease 2019 (COVID-19), medRxiv, 11, 827 (2020).

Allergies are reactions of the immune system toward foreign substances that do not cause immunogenicity under healthy conditions. Accumulating evidence indicates that T helper type 2 cell-derived cytokines such as interleukin 4 (IL-4), interleukin 5 (IL-5), and interleukin 13 (IL-13) play critical roles in orchestrating and amplifying allergic inflammation in asthma. See, e.g., Nakajima H, Takatsu K., Role of Cytokines in Allergic Airway Inflammation, Int. Arch. Allergy Immunol., 142 (4), 265-73 (2007).

Vulvodynia is chronic pain or discomfort around vulva for at least three months. The vulvar pain syndrome (VPS) is a multifactorial disease severely influencing the lifestyle of affected women. Eleven molecules, specifically involved in the pro-inflammatory pathway have been shown to be significantly modulated in VPS patients in comparison to healthy women, suggesting a persistent inflammatory process. See Nunzia Zanotta et al., Cytokine Profiles of Women with Vulvodynia: Identification of a Panel of Pro-inflammatory Molecular Targets, Eur. J Obstet. Gynecol. Reprod. Biol., 226, 66-70 (2018).

Otitis media is inflammation or infection in the middle ear. Otitis media can occur as a result of a cold, sore throat, or respiratory infection. Acute otitis media (AOM) is an inflammatory response to microbes in the middle ear, sometimes associated with ruptures of the tympanic membrane. Strikingly higher levels of IL-1β, tumour necrosis factor (TNF), interleukin 8 (IL-8) and interleukin 10 (IL-10) in culture-positive than in culture-negative middle ear effusions. S. Skovbjerg et al., High Cytokine Levels in Perforated Acute Otitis Media Exudates Containing Live Bacteria, European Society of Clinical Microbiology and Infectious Diseases, CMI, 16, 1382-1388 (2010).

Inflammatory bowel disease (IBD), which includes Crohn's disease (CD) and ulcerative colitis (UC), represents a group of chronic disorders characterized by inflammation of the gastrointestinal tract, typically with a relapsing and remitting clinical course. Cytokines play a key role in IBD that determine T cell differentiation of Th1, Th2, T regulatory and newly described Th17 cells. Cytokine levels in time and space orchestrate the development, recurrence and exacerbation of the inflammatory process in IBD. Therefore, several cytokine therapies have been developed and tested for the treatment of IBD patients. Fausto Sanchez-Muñoz et al., Role of Cytokines in Inflammatory Bowel Disease, World J. Gastroenterol 14 (27), 4280-4288 (2008).

Myasthenia gravis (MG) is a chronic autoimmune disorder in which, antibodies destroy the communication between nerves and muscle, resulting in weakness of the skeletal muscles. Inflammation could be a key player for understanding the pathogenesis of Myasthenia gravis. In patients with MG, serum levels of a proliferation-inducing ligand (APRIL), interleukin 19 (IL-19), interleukin 20 (IL-20), interleukin 28A (IL-28A) and interleukin 35 (IL-35) were significantly increased as compared with controls (p<0.05). Among them, IL-20, IL-28A and IL-35 were significantly decreased after treatment (p<0.05). Uzawa, A. et al., Changes in Inflammatory Cytokine Networks in Myasthenia Gravis., Sci. Rep. 6, 25886, doi: 10.1038/srep25886 (2016).

Multiple sclerosis (MS) is a chronic autoimmune disease that affects the central nervous system and is characterized by demyelination, axonal loss, gliosis and inflammation. Immune system cells mistakenly attack myelin in the central nervous system (e.g., brain, optic nerves, and spinal cord) that coats nerve fibers (axons). Proinflammatory cytokines such as interleukin-17 (IL-17), interleukin-22 (IL-22), TNF-α, interleukin 1 (IL-1), interleukin 12 (IL-12) and interferon-γ (IFN-γ) may cause MS through several signaling pathways. Conversely, anti-inflammatory circulating cytokines such as IL-4 and IL-10 are reduced and can exert a direct protective effect in this condition. Kexin Wang et al., The Properties of Cytokines in Multiple Sclerosis: Pros and Cons, Am. J. Med. Sci., 356 (6), 552-560 (2018).

Chronic alcohol consumption leads to hepatocellular injury and liver inflammation. Alcohol consumption promotes liver inflammation by increasing translocation of gut-derived endotoxins to the portal circulation. Inflammatory cytokines, such as TNF-α and IFN-γ, have been shown to induce liver injury in the rat model of alcoholic liver disease (ALD). Hepatoprotective cytokines, such as IL-6, and anti-inflammatory cytokines, such as IL-10, are also associated with ALD. Hideto Kawaratani et al., The Effect of Inflammatory Cytokines in Alcoholic Liver Disease, Mediators Inflamm., 2013:495156 (2013).

Mucositis is a toxic side effect of anti-cancer treatments with radiation and chemotherapy. Pro-inflammatory cytokines have been implicated in the pathophysiology of chemotherapy-induced gastrointestinal mucositis [Ong, Z. Y., Gibson, R. J., Bowen, J. M. et al. Pro-inflammatory cytokines play a key role in the development of radiotherapy-induced gastrointestinal mucositis. Radiat Oncol 5, 22 (2010)].

Sepsis is the body's overwhelming and life-threatening response to infection that can lead to tissue damage, organ failure, and death. The pro-inflammatory cytokines are closely related with the progression of the coagulation process in sepsis. Certain inflammatory skin diseases have been associated with overproduction of cytokines, alteration in cytokine receptors, or dysregulation of cytokines. Among the most frequent chronic inflammatory skin diseases are atopic dermatitis, psoriasis, urticaria, lichen planus, and hidradenitis suppurativa, driven by a complex interplay of genetics and environmental factors. Autoimmunity is another important cause of chronic skin inflammation.

A chronic pro-inflammatory status is a pervasive feature of aging leading to degeneration of several organs. There is strong evidence that the development of age-related multi-factorial conditions such as cancer, cardiovascular disease, Alzheimer's disease, type II diabetes, frailty, sarcopenia, and osteoporosis is associated with low-grade elevations of circulating inflammatory mediators.

In contrast to young individuals, aged individuals have consistently elevated levels of inflammatory cytokines, especially interleukin-6 (IL-6) and tumor necrosis factor-α (TNF-α). Singh T, Newman A B., Inflammatory Markers in Population Studies of Aging., Ageing Res. Rev. 10 (3), 319-29 (2011).

Renal damage results in accumulation of unsafe levels of extra cellular matrix. This is known as kidney (or renal) failure. Unless treated, this can cause death.

There are 2 main types of kidney (renal) failure: acute (sudden), also known as “acute kidney injury” (AKI); and chronic (over time), also known as chronic kidney disease (CKD). Chronic kidney disease (CKD) is defined as the presence of structural or functional abnormalities in one or both kidneys that have been present for an extended period of time. CKD is a worldwide public health problem affecting approximately 8-10% of the population in western countries. 30 million people or 15% of US adults are estimated to have CKD. Prevalence of CKD in patients with type 2 diabetes is currently around 40% and continues to grow.

CKD is also common in cats. The prevalence of CKD also increases with age. More than 10% of dogs and 30% of cats over 15 years of age have been found to be diagnosed with CKD.

Excess accumulation of extracellular matrix (primarily composed of collagen) is called renal fibrosis, and it usually results in loss of function when a normal tissue is replaced with a scar tissue. Since unchecked renal fibrosis precedes irreversible kidney damage, halting or slowing the insidious progression of renal fibrosis is an ideal treatment target for preventing deterioration of CKD to end-stage kidney disease.

Although there is no definitive cure for CKD, treatment can improve and prolong the lives of mammals with this disease. Therapy is geared towards minimizing the buildup of toxic waste products in the bloodstream, maintaining adequate hydration, addressing disturbances in electrolyte concentration, supporting appropriate nutrition, controlling blood pressure, and slowing the progression of the kidney disease.

Kidney disease treatments might include high blood pressure medications (AC-inhibitors or angiotensin II receptor blockers) to preserve kidney function, diuretics, medications to treat anemia, hormone erythropoietin, and medications to lower cholesterol levels.

Despite ongoing research, there is currently no effective treatment that significantly slows the progression of renal fibrosis in humans or cats. Jack Lawson et al., Renal Fibrosis in Feline Chronic Kidney Disease: Known Mediators and Mechanisms of Injury, The Veterinary Journal, 203, 18-26 (2015).

The association between oxidative stress, inflammation, and CKD progression is well established. Reducing oxidative stress and inflammation are attractive targets to slow down CKD progression.

Pentacyclic triterpenoids such as ursolic acid and oleanolic acid are believed to have a wide range of therapeutic benefits including anti-oxidant, anti-inflammatory, anti-cancer, anti-allergic, hepatoprotective, gastroprotective, hypolipidemic, hypoglycemic, lipolytic anti-obesity, anti-atherogenic and immunomodulatory effects.

With antioxidant and anti-inflammatory properties, ursolic acid is a good candidate to manage renal health.

Traditionally fruits of Randia echinocarpa (granjel) have commonly been employed in the treatment of kidney ailments in Mexico. Key components of Randia echinocarpa are identified as ursolic acid and oleanolic acid. Robert Bye et al., Ethanobotanical and Phytochemical Investigation of Randia echinocarpa, Anales Inst. Biol. Univ. Nac. Auton. Mexico. Ser. Bot., 62 (1), 87-106 (1991).

Transforming growth factor beta 1 (TGF-β1) has been reported to be related to various diseases including chronic glomerulonephritis and diabetic nephropathy. Previous in vitro studies with Balb/c 3T3 cells reported in, e.g., JP 2000159673 and by Hiromitsu Yoshimura et al., described TGF-β1 inhibitory action by an extract of Clerodendranthus spicatus containing ursolic and oleanolic acids. Hiromitsu Yoshimura et al., In Vitro TGF-β1 Antagonistic Activity of Ursolic and Oleanolic Acids Isolated from Clerodendranthus spicatus, Planta. Med. 69 (7), 673-675 (2003). The concentration of ursolic acid that inhibits the specific binding of TGF-β 1 by 50% was 3.0 μg/ml. Id.

From a physico-chemical property aspect, ursolic acid is the ursane-type pentacyclic triterpenoid, and is a white crystalline, odorless, tasteless, and water-insoluble compound.

Ursolic acid is a pentacyclic triterpene compound, with the molecular formula of C30H48O3 and a molecular weight of 456.68 g/mol. It is found in plants, including Salvia officinalis, Salvia miltiorrhiza L, Eriobotrya japonica (Loquat), Rosmarinus officinalis, Origanum onites, Origanum vulgare, Ocimum accessions, Ocimum sanctum, Oldenlandia diffusa, Olive pulp, Macrocarpium officinale, Prunella vulgaris, Psychotria serpens, Hyptis capitata, Hedyotis corymbose, Swertia chirata, Hedyotis diffusa, and Vitex negundo.

Triterpene family include lupane, oleanane and ursane, arjunolic acid, maslinic acid, lupenol, betulin, betulinic acid, asiatic acid, corosolic acid, boswellic acid, glycyrrhetinic acid, madecassic acid, and more.

Triterpenes isolated from the mushroom Ganoderma lucidum have shown to markedly suppress the secretion of inflammatory cytokine tumor necrosis factor-α (TNF-α) and interleukin-6 (IL-6). Shailesh Dudhgaonkar et al., Suppression of the Inflammatory Response by Triterpenes Isolated from the Mushroom Ganoderma Lucidum, Int. Immunopharmacol., 9:11, 1272-1280 (October 2009).

AnaMarquez-Martin et al. investigated the effect of minor components of olive pomace oil on pro-inflammatory cytokine production by human peripheral blood mononuclear cells. They reported that pentacyclic triterpenes in orujo oil exhibited pro- and anti-inflammatory properties depending on chemical structure and dose, and concluded that they may be useful in modulating the immune response. AnaMarquez-Martin et al., Modulation of Cytokine Secretion by Pentacyclic Triterpenes from Olive Pomace Oil in Human Mononuclear Cells., Cytokine, 36:5-6, 211-217 (2006).

Study results have indicated that betulin and betulinic acid inhibited ethanol-induced activation of hepatic stellate cells (HSCs) on different levels, acting as antioxidants, inhibitors of cytokine production, and inhibitors of TGF-β, and NFκB/IκB transduction signaling. Betulin and betulinic acid attenuate ethanol-induced liver stellate cell activation by inhibiting reactive oxygen species (ROS), cytokine (TNF-α, TGF-β) production, and by influencing intracellular signaling. AgnieszkaSzuster-Ciesielska et al., Betulin and Betulinic acid Attenuate Ethanol-induced Liver Stellate Cell Activation by Inhibiting Reactive Oxygen Species (ROS), Cytokine (TNF-α, TGF-β) Production and by Influencing Intracellular Signaling, Toxicology, 280:3, 152-163 (2011).

Yue Huang et al. reported that a cranberry extract (containing ursolic acid) inhibited the NF-κB transcriptional response in human T lymphocytes, LPS-stimulated release of IL-1β, IL-6, and TNF-α in PBMs, and the catalytic activity of COX-2. Yue Huang et al., Effects of Cranberry Extracts and Ursolic acid Derivatives on P-fimbriated Escherichia Coli, COX-2 Activity, Pro-inflammatory Cytokine Release and the NF-κB Transcriptional Response in Vitro, Pharmaceutical Biology, 47:1, 18-25 (2009).

Hai Bing Peng et al. reported that protective effects of oleanolic acid were due, at least in part, to its anti-oxidant activity and its ability to decrease the expression of cytokines and collagen by modulating the AKT/NF κB pathway. Peng H B et al., Protective Effects of Oleanolic Acid on Oxidative Stress and the Expression of Cytokines and Collagen by the AKT/NF κB Pathway in Silicotic Rats, Mol. Med. Rep., 15 3121-3128 (2017).

Ursolic acid and oleanolic acid have a wide range of therapeutic benefits including anti-oxidant, anti-inflammatory, anti-cancer, anti-allergic, hepatoprotective, gastroprotective, hypolipidemic, hypoglycemic, lipolytic anti-obesity, anti-atherogenic and immunomodulatory effect.

Previous in vitro studies with Balb/c 3T3 cells reported in, e.g., JP 2000159673 and by Hiromitsu Yoshimura et al., described TGF-β1 inhibitory action by an extract of Clerodendranthus spicatus containing ursolic and oleanolic acids. Hiromitsu Yoshimura et al., In Vitro TGF-β1 Antagonistic Activity of Ursolic and Oleanolic Acids Isolated from Clerodendranthus spicatus, Planta. Med. 69 (7), 673-675 (2003). The concentration of ursolic acid that inhibits the specific binding of TGF-β 1 by 50% was 3.0 μg/ml. Id.

Mapanga et al. reported in vivo evidence that oleanolic acid extracted from Syzygium cordatum leaves ameliorates kidney function in diabetic rats. The rats were caged and treated twice daily with oleanolic acid (60 mg/kg, p.o.) for five weeks. Oleanolic acid was added to the infusate at 90 ug/h for 1.5 h (treatment period), resulting in a total dose of 0.45 mg/kg (for a 300 g rat). Separate groups of non-diabetic and diabetic rats were treated with oleanolic acid (60 mg/kg, p.o.) twice every third day for five weeks. Mapanga et al. Renal Effects of Plant-Derived Oleanolic Acid in Streptozotocin-Induced Diabetic Rats., Renal Failure, 31, 481-491 (2009).

CN101732323 describes the usage of ‘low-dose’ ursolic acid as medicament for treating diabetic early nephropathy. Oral dose employed in the study was reported as of 50-70 mg/kg weight and this was described as ‘low dose’. For an average person of 80 kilos, this ‘low dose’ will be about 4,000 to 5,600 mg; which is considered very high.

The reno-protective effect of ursolic acid in gentamicin-induced renal damage in Wistar albino rats has been reported. In this study, pure ursolic acid (Sigma Aldrich Chemicals Pvt. Ltd., UK) was dissolved in 14% DMSO, and administered orally in a dose of 2 mg/kg, 5 mg/kg, and 10 mg/kg. Preethi G. Pai et al., Nephroprotective Effect of Ursolic Acid in a Murine Model of Gentamicin-Induced Renal Damage, International Scholarly Research Network (ISRN) Pharmacology, Article ID 410902 (2012).

Pure ursolic acid (30 mg/kg dose) of suspended in 0.5% CMC solution orally was tested in rats for 28 days with simultaneous adenine feeding. Richa Thakur et al. Ameliorative Effect of Ursolic Acid on Renal Fibrosis in Adenine-Induced Chronic Kidney Disease in Rats, Biomedicine & Pharmacotherapy, 101 972-980 (2018).

Other studies have been conducted to show the effects of ursolic acid. See, e.g., Qi My et al., Study on the Protective Effect of Ursolic Acid on Alloxan-induced Diabetic Renal Injury and its Underlying Mechanisms, Chinese J. Applied Physiology, 30 (5), 445-448 (2014), at https://europepmc.org/article/med/25571638 (Ursolic acid was fed at a dose of 35 mg/kg/d, i.g. continuously for 8 weeks); Zhaohui Jia, Ursolic Acid Treats Renal Tubular Epithelial Cell Damage Induced by Calcium Oxalate Monohydrate via Inhibiting Oxidative Stress and Inflammation, Bioengineered, 12:1, 5450-5461 (2021) (Rats were daily given 40 mg/kg of ursolic acid via gavage).

Ursolic acid treatment has been shown to effectively reduce the urinary albumin/creatinine ratio (p<0.05) in mice. Tian-Kui Ma et al., Ursolic Acid Treatment Alleviates Diabetic Kidney Injury by Regulating the ARAP1/AT1R Signaling Pathway, Diabetes Metabolic Syndrome and Obesity, 12, 2597-2608 (2019). There, ursolic acid (UA) treatment group of mice were fed a diet containing 0.3% UA (0.3 g of UA per 100 g of standard feed) for 10 weeks.

High-dose ursolic acid (50 mg/kg) and low-dose ursolic acid (25 mg/kg) have been shown to have significant protective effects on diabetic rats. Hui-lin Xu, Ursolic Acid Improves Diabetic Nephropathy via Suppression of Oxidative Stress and Inflammation in Streptozotocin-induced Rats, Biomedicine & Pharmacotherapy, 105, 915-921 (2017).

There are references disclosing studies claiming protective effects of ursolic acid, albeit at very high dosages as 25 to 70 mg/kg body weight. This translates to usage of 4 to 5 grams of ursolic acid for an average person per day. Considering the extraction and concentration and processing of ursolic acid, this is commercially not feasible. Moreover, such high dosage may have unintended side effects on other systems of the body. There is a need to decrease an effective dose.

In other reported studies, either ursolic acid was tested by dissolving in DSMO, which cannot be clinically practiced. In other studies, ursolic acid was delivered as a parenteral dose. Ursolic acid was dissolved in a solvent and directly injected to the body.

According to the Biopharmaceutical Classification System (BCS), drug substances are classified to four classes upon their solubility and permeability characteristics. Of these classes, Class IV compounds are low permeable and low soluble, and hence are poorly bioavailable. Usually, these compounds are not well absorbed over the intestinal mucosa and a high variability is expected.

Ursolic acid belongs to BCS Class IV category due to its low aqueous solubility (<5.64 μg/mL) (Lin. H. et al., Determination of Equilibrium Solubility and Apparent Oil/Water Partition Coefficient of Ursolic Acid. Chin. J. Mod. Appl. Pharm., 29, 635-637 (2012)) and low permeability (Papp=2.8 Å~10-6 cm/s in the apical-to-basolateral direction at 20 μM) (Qiang. Z. et al. Permeability of Rosmarinic Acid in Prunella Vulgaris and Ursolic Acid in Salvia Officinalis Extracts Across Caco-2 Cell Monolayers., J. Ethnopharmacol. 137, 1107-1112 (2011)).

Only about 0.6% of ingested ursolic acid was recovered in plasma in rats after administrated orally. Liao. Q. et al., LC-MS Determination and Pharmacokinetic Studies of Ursolic Acid in Rat Plasma after Administration of the Traditional Chinese Medicinal Preparation Lu-Ying Extract, Yakugaku Zasshi, 125, 509-515 (2005).

Solid oral dose is generally the most preferred method for any therapeutics because of low manufacturing cost and convenience to the user, and hence there is a need for low and effective dose of ursolic acid.

Ursolic acid is water-insoluble and is in stable crystalline form. It can be converted into an amorphous form for better therapeutic functions. However, the non-crystalline state is thermodynamically unstable, and there is a tendency to entropically drive ursolic acid in the non-crystalline state to a stable crystalline state. The high internal energy and specific volume of the amorphous state can lead to enhanced dissolution and bioavailability, but can also create the possibility that during processing or storage, the amorphous state spontaneously converts back to the crystalline state. See Huttenrauch R., Molecular Pharmaceutics as a Basis for Modern Drug Formulation, Acta Pharmaceutica Technology Supplement, 24 (6), 55-127 (1978); Yoshioka M et al., Crystallization of Indomethacin from the Amorphous State Below and Above its Glass Transition Temperature, J. Pharmaceutical Sciences, 83 (12), 1700-5 (1994).

On the other hand, Danni Yu et al. reported that crystalline ursolic acid exhibited poor physical stability and it underwent structural change after 30 days of storage under 25° C./75% RH or 40° C./75% RH.

Danni Yu et al. and CN102234304A found that amorphous ursolic acid prepared by ball milling exhibited a nearly complete conversion toward its stable crystalline form after 8 days of storage at both 25° C./75% RH or 40° C./75% RH. Danni Yu et al., Triple Strategies to Improve Oral Bioavailability by Fabricating Co-amorphous Forms of Ursolic Acid with Piperine: Enhancing Water-Solubility, Permeability and Inhibiting Cytochrome P450 Isozymes, Mol. Pharmaceutics, 17 (12): 4443-4462 (2020). Having Piperazine limits Ursolic acid's therapeutic application and hence not useful for all practical purposes.

Other techniques were also applied to improve pharmacological properties of ursolic acid. Some of them include liposome formulations, encapsulation with hydroxypropyl β cyclodextrin and nanostructured lipid carriers, self-nano emulsified formulations, phospholipid complexes and solid dispersions, etc. Challenges in scaling up these laboratory processes, usage of solvents in certain processes, pose practical problems.

There is no universal or reliable method to select a technology or a polymer to have guaranteed amorphous stability and improved bioavailability.

Although ursolic acid showed significant biological activity in in vitro assays and in some animal models, determination of in vivo efficacy of ursolic acid in humans has been difficult. Some of the reasons are their poor biological activity, low solubility, low absorption, low permeability, first pass metabolism, and poor metabolism, pre-systemic excretion from the intestine or liver.

There is a need for a practical way to improve the therapeutic function of ursolic acid.

### BRIEF SUMMARY OF THE INVENTION

The present invention provides new and improved methods to prepare stable amorphous pentacyclic triterpenoid compounds, a composition prepared with such amorphous pentacyclic triterpenoid, and a method to mitigate effects caused by pro-inflammatory cytokines in mammals, including humans, by providing an effective dose of a stable amorphous pentacyclic triterpenoid. The present invention also provides an effective low-dose therapeutic composition prepared with a stable amorphous pentacyclic triterpenoid.

In some aspects, the preset invention provides a pharmaceutical composition for mammals, including humans and companion animals, particularly to treat renal damage.

In a specific embodiment, the present invention provides a method of treating renal damage in mammals, including companion animals, comprising administering to a subject in need of such treatment a pharmaceutical composition prepared with an amorphous pentacyclic triterpenoid, such as ursolic acid.

In some embodiments, the pharmaceutical composition has been prepared by a process comprising the steps of: blending a pentacyclic triterpenoid and a carrier such as an inert water-soluble polymer having a glass transition temperature lower than 170° C.; melt extruding the blend at a temperature between about 60° C. to about 180° C.; grinding the extrudate; blending the ground extrudate with excipients to form the pharmaceutical composition.

In some embodiments, the pharmaceutical composition is capsule, tablet, chewable tablet, soft chew, gummy, lozenge, or powder, and such composition is free from any residual solvent. In some embodiments, the process according to the invention irreversibly converts a pentacyclic triterpenoid to amorphous form having enhanced aqueous solubility.

### BRIEF DESCRIPTION OF THE DRAWINGS

FIG. 1 shows X-ray diffractometry of: [A] unprocessed ursolic acid indicating crystalline nature; [B] processed sample of ursolic acid (active ingredient 1) indicating amorphous nature; [C] processed sample of ursolic acid (active ingredient 2) indicating amorphous nature.

FIG. 2 shows the amorphous form of ursolic acid samples stored for 3 to 15 months. Ursolic acid is stable for the tested 15 months, and no reversal of amorphous to crystalline form proves the merit of this invention.

### DETAILED DESCRIPTION OF THE INVENTION

In one aspect, the present invention provides a dispersion composition in the form of a solid dispersion comprising an amorphous pentacyclic triterpenoid and a carrier. In some embodiments, the pentacyclic triterpenoid is ursolic acid and the carrier is a water-soluble polymer having a glass transition temperature lower than 170° C.

In another aspect, the present invention provides a method for preparing a solid dispersion comprising an amorphous pentacyclic triterpenoid, such as ursolic acid, by blending a pentacyclic triterpenoid with a carrier, such as a water-soluble polymer having glass transition temperature lower than 170° C. In some embodiments, the dispersion is prepared by melt extrusion.

In some embodiments, a pentacyclic triterpenoid as used according to the subject invention comprises ursolic acid, oleanolic acid, arjunolic acid, maslinic acid, lupenol, betulin, betulinic acid, asiatic acid, corosolic acid, boswellic acid, glycyrrhetinic acid, madecassic acid, or a combination thereof. The pentacyclic triterpenoids may be in the form of pharmaceutically acceptable salts, isomers, solvates, and/or extracts or a combination thereof. In a specific embodiment, the pentacyclic triterpenoid is ursolic acid.

Ursolic acid is a naturally-derived lipophilic pentacyclic triterpene acid whose chemical formula is C₃₀H₄₈O₃, and a melting point of 283-285° C. In the present invention, ursolic acid may be in the form of a pharmaceutically acceptable salt, isomer, or solvate, or an extract containing ursolic acid.

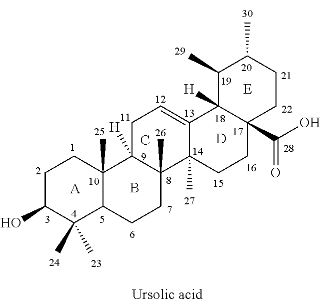

In some embodiments, the present invention provides a pharmaceutical composition as a final composition prepared with a solid dispersion of an amorphous pentacyclic triterpenoid. such as ursolic acid. as an active ingredient and a method of making same. The solid dispersion of an amorphous pentacyclic triterpenoid may be formulated into a pharmaceutical composition by itself or formulated with one or more excipients into a pharmaceutical composition. In specific embodiments, the pentacyclic triterpenoid as an active ingredient is ursolic acid.

In some embodiments, the pentacyclic triterpenoid in an amorphous solid dispersion is therapeutically effective at about 0.05 mg to about 20 mg/kg body weight, preferably at about 0.5 mg to about 10 mg/kg body weight, and more preferably at 1 mg to about 5 mg/kg body weight.

In some embodiments, a pharmaceutical composition according to the present invention is used in a method for treating a medical condition caused by overactive pro-inflammatory cytokines. In specific embodiments, such medical condition is kidney disease, acute kidney injury, or chronic kidney disease, a morbidity due to Covid-19 viral infection, a morbidity due to virus infection, a morbidity due to bacterial infection, tuberculosis, diabetic nephropathy, inflammatory arthritis, allergy or allergic asthma, periodontal disease, vulvodynia, otitis, ulcerative colitis, Crohn's disease, Myasthenia gravis, multiple sclerosis, liver disease, alcoholic liver disease, mucositis, major Depressive Disorder, Anxiety-related disorder, aging, or chronic inflammatory skin disease.

In some embodiments, a pharmaceutical composition according to the present invention is administered orally, ingested as a therapeutic supplement, as an adjunct to food or feed.

In some embodiments, the solid dispersion of an amorphous pentacyclic triterpenoid according to the present invention renders the pentacyclic triterpenoid more soluble in water and/or therapeutically more effective, so that the effective dosage of the pentacyclic triterpenoid is reduced compared to conventional formulations of the pentacyclic triterpenoid. In some embodiments, such low dose formulation helps reduce renal damage.

In some embodiments, a pharmaceutical composition according to the present invention is used in a method for reducing renal damage in a subject, wherein said subject suffers from a renal condition such as a CKD and/or said subject is at risk for developing such renal condition. As used herein, renal damage refers to oxidative stress and/or inflammation in a subject. Oxidative stress and inflammation can be measured by methods known in the art.

In some embodiments, an amorphous solid dispersion according to the present invention comprises a pentacyclic triterpenoid as an active ingredient, preferably in the amount of 10 to 70 wt % based on the total weight of the final composition. Because of low density, at 10 wt % level, it is not difficult to achieve content uniformity in the blend. In some embodiments, the pentacyclic triterpenoid is ursolic acid.

In some embodiments, an amorphous solid dispersion according to the present invention further comprises a carrier. The carrier may include a water-soluble polymer having a glass transition temperature below 170° C., which according to some embodiments comprises Polyvinyl caprolactam, polyvinyl acetate-polyethylene glycol graft copolymer (Soluplus®), polyvinylpyrrolidone-vinyl acetate copolymer (PVP VA64), polyethylene glycol (PEG), Eudragit® EPO, and/or hypromellose acetate succinate (HPMCAS). In some embodiments, the water-soluble polymer is contained in the amount of 30 wt % or more, more preferably in the amount of 30 to 90 wt %, based on the total weight of the final composition. In some embodiments, polyvinyl pyrrolidone having any molecular weight can be used, but in particular those having the molecular weight of 45,000 to 70,000 are desired since they may build the viscosity needed for melt-extrusion. More preferable polymer is vinylpyrrolidone-vinyl acetate copolymer. Other polymers can also be used according to the present invention.

In some embodiments, an amorphous solid dispersion according to the present invention further comprises a plasticizer. The usage of a plasticizer such as D-alpha-tocopheryl polyethylene glycol 1000 succinate (TPGS), polyethylene glycol 400, and other customary plasticizers are not restricted, to the extent that the use does not affect the amorphous nature of a pentacyclic triterpenoid such as ursolic acid. In some embodiments, an amorphous solid dispersion according to the present invention does not comprise a plasticizer.

In some embodiments, an amorphous solid dispersion according to the present invention comprises a pentacyclic triterpenoid (such as ursolic acid) in the amount of 5 to 50 wt % and a water-soluble polymer (such as polyvinyl pyrrolidone) in the amount of 5 to 95 wt % with respect to the final composition.

In some embodiments of a method according to the invention, a blend comprising a pentacyclic triterpenoid (such as ursolic acid) and a water-soluble polymer is melted when it passes through at least four (4) heating zones whose temperature is sequentially lowered. Specifically, the blend which has been mixed in advance as a powder is introduced into an extruder and melt-extruded to prepare the solid dispersion of the present invention wherein the extruder is made of several heating zones designed to be distinguished from each other, and which are connected in series. In some embodiments, the distinguished heating zones are controlled to have a temperature lower than the melting point of the pentacyclic triterpenoid (e.g., ~284° C.).

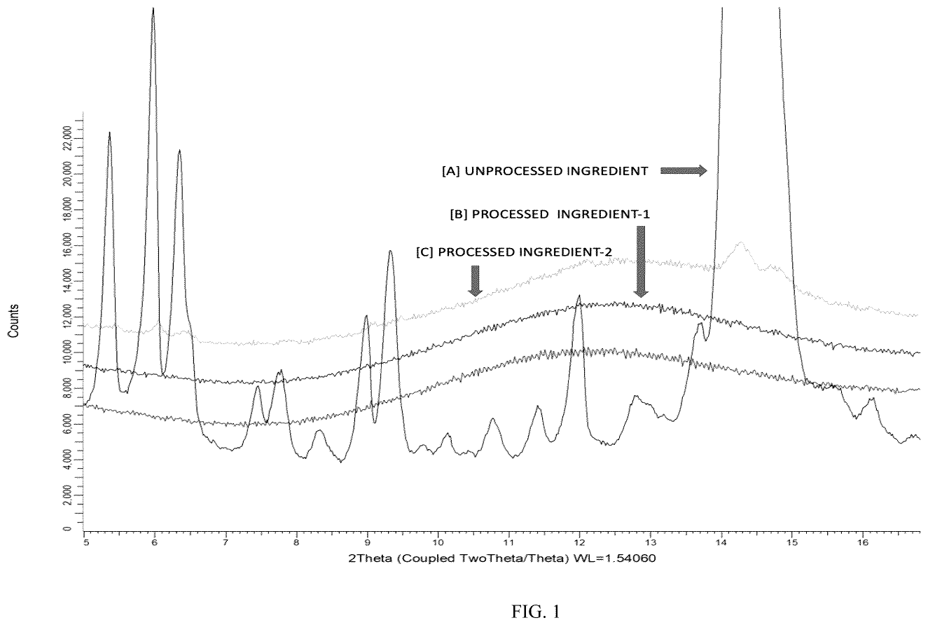

Although the melting of the blend is performed at a temperature lower than the melting point of the pentacyclic triterpenoid, the extrusion is performed through the specific sustained cooling and sequential melting by passing through the several heating zones whose setting temperatures are lowered sequentially causing changes to the active ingredient, causing a conversion of crystalline structure to amorphous form of the pentacyclic triterpenoid. In some embodiments, melt extrusion is performed at a temperature of about 60° C. to about 180° C. As illustrated in the FIG. 1, the active ingredient ursolic acid processed according to the method of the present invention described herein clearly shows its conversion to amorphous form.

The melt extruded product is ground to fine powder by using a mill, blended with excipients to form a pharmaceutical composition in the form of, e.g., a tablet, capsule, chewable tablet, soft chew, gummy, bar, oral paste, lozenge, or powder. In some embodiments, such pharmaceutical composition is free of solvent.

Excipients that may be used to prepare a pharmaceutical composition according to the present invention include, but are not limited to, flow agents to fill in to oral capsules, tableting agents, disintegrants to form tablets, flavoring agents, sweeteners to form chews, and solidified fats to form suppositories.

Table 1 below summarizes processing conditions and measured parameters of a dispersion composition according to the present invention.

TABLE 1
Processing conditions and measured parameters

| Parameters | | |
|---|---|---|
| Extruder | Steer Omega 20P | |
| B 1 | Ambient | |
| B 2 | 30° | C. |
| B 3 | 60° | C. |
| B 4 | 120° | C. |
| B 5 | 180° | C. |
| B 6 | 180° | C. |
| B 7 | 180° | C. |
| B 8 | 180° | C. |
| Open die head | 180° | C. |
| Screw speed (RPM) | 450 | |
| Torque (%) | 61-71 | |
| Feed rate | 5 | kg/hr |

| | |
|---|---|
| Extrudate | Clear |

### An Exemplary Method for Carrying Out the Invention

Method of manufacturing: An extract containing ursolic acid is mixed with vinylpyrrolidone-vinyl acetate copolymer at a ratio of 10:90 to form a uniform physical mixture (PM). This PM was passed through a hot melt extruder (Thermofisher, Steer) at temperature 180° C. This extrudate is collected from the die and cooled at ambient temperature and pulverized to 80 mesh to obtain an extrudate material as an amorphous solid dispersion.

The extrudate material was tested to confirm the conversion of crystalline structure of ursolic acid to an amorphous form, and this amorphous state was maintained during storage as described herein.

Solubility of the extrudate material was compared with untreated material comprising ursolic acid and found to be substantially improved over the untreated material.

This extrudate material is formulated into a pharmaceutical composition in the form of, e.g., a capsule, tablet, chewable tablet, soft chew, gummy, lozenge, delivering desired amounts of ursolic acid as an active ingredient in the pharmaceutical composition prepared according to the present invention.

### Product Testing

### 1. Conversion to Amorphous Form:

FIG. 1: X-ray diffractometry of: [A] unprocessed ursolic acid exhibiting crystalline nature; [B] processed ursolic acid (active ingredient 1) exhibiting amorphous nature; [C] processed ursolic acid (active ingredient 2) exhibiting amorphous nature.

### 2. Stability of Amorphous Form During the Shelf-Life Study:

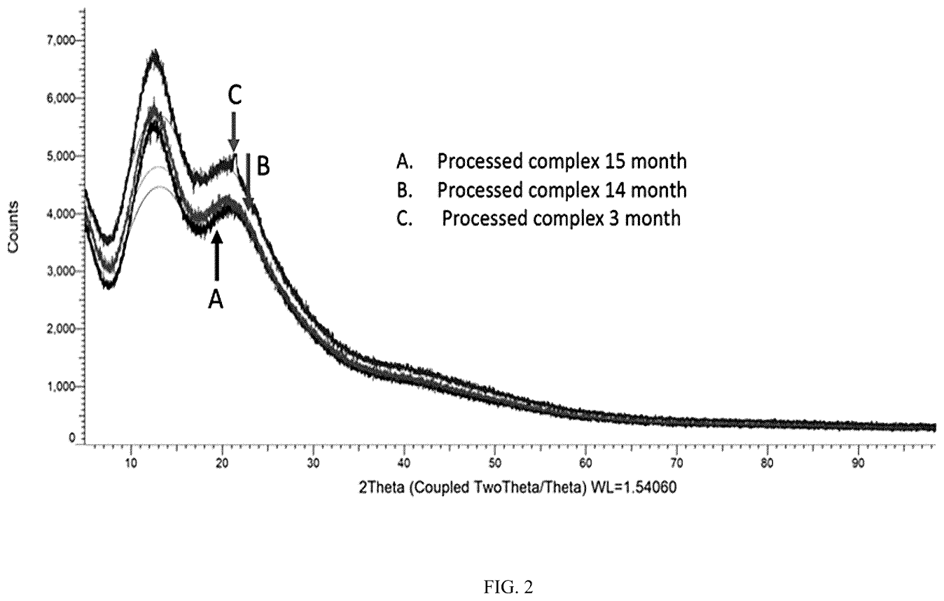

FIG. 2: Shows the amorphous form of ursolic acid samples stored for 3 to 15 months. Ursolic acid is stable for the tested 15 months, and no reversal of amorphous to crystalline form proves the merit of this invention.

### 3. Low Dose is Therapeutically Effective:

A sprinkler formula for cats was prepared with sage extract containing ursolic acid and oleanolic acid processed according to the present invention and with powdered palatant as a filler. Level scoop of 0.625 CC volume delivers about 400 mg of mix providing 5 mg of ursolic acid. This is the dose for a 5 kilo cat. Two servings per day providing a total of 2 mg/kg dose was found to be therapeutically effective in managing renal health of the cat.

The active ingredient ursolic acid processed according to a method of the present invention (1) is thermally stable as an amorphous solid dispersion (2) is encapsulated at molecular level to form stable amorphous form without any recrystallization during storage (3) has improved solubility (4) exhibits consistent results in repeated experiments. This validates the value of present process of preparing a stable and highly water-soluble form of a pentacyclic triterpenoid, which hence is therapeutically effective at lower dose.

The term “subject” or “patient,” as used herein, describes an organism, including mammals such as primates. Mammalian species that can benefit from the disclosed invention include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters.

As used herein, the term “effective amount” or “effective dose” is used to refer to an amount of something (e.g., a compound, a composition, time) that is capable of causing a desired outcome (e.g., improvement in the consistency of the final composition).

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention, e.g., the ability to improve the bioavailability of a substance. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example, within 2 standard deviations of the mean. As further examples, “about” can be understood as within 10%, 9%, 8%, 7%, 6%, 5%, 4%, 3%, 2%, 1%, 0.5%, 0.1%, 0.05%, or 0.01% of the stated value.

When ranges are used herein, such as for dose ranges, combinations and sub-combinations of ranges (e.g., subranges within the disclosed range), specific embodiments therein are intended to be explicitly included.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

1. A process of preparing the pharmaceutical or therapeutic supplement composition of claim 9 comprising a pentacyclic triterpenoid, wherein the process comprises:
a) blending the pentacyclic triterpenoid, and a water-soluble polymer having a glass transition temperature lower than 170° C. to obtain a mixture;
b) melt extruding the mixture at a temperature from about 60° C. to about 180° C. to obtain an extrudate;
c) grinding the extrudate to obtain a ground extrudate comprising a solid dispersion of an amorphous form of the pentacyclic triterpenoid; and
d) formulating the solid dispersion to obtain the pharmaceutical or therapeutic supplement composition.

2. The process of claim 1, wherein the pentacyclic triterpene is selected from the group consisting of ursolic acid, oleanolic acid, arjunolic acid, maslinic acid, lupenol, betulin, betulinic acid, asiatic acid, corosolic acid, boswellic acid, glycyrrhetinic acid, and madecassic acid.

3. The process of claim 1, wherein the pentacyclic triterpenoid is ursolic acid, or oleanolic acid.

4. The process of claim 1, wherein the pentacyclic triterpenoid is about 5% to about 50% by the weight of the pharmaceutical or therapeutic supplement composition.

5. The process of claim 1, wherein the water-soluble polymer is selected from the group consisting of polyvinyl caprolactam, polyvinyl acetate-polyethylene glycol graft copolymer, polyvinylpyrrolidone-vinyl acetate copolymer, polyethylene glycol, Eudragit® EPO, and hypromellose acetate succinate.

6. The process of claim 1, wherein the water-soluble polymer is a poly vinylpyrrolidone-vinyl acetate copolymer.

7. The process of claim 1, wherein the water-soluble polymer is present in a range of from about 5% to about 95% by weight of the pharmaceutical or therapeutic supplement composition.

8. The process of claim 1, wherein the mixture of the pentacyclic triterpenoid and the water-soluble polymer in the melt extruding b) is melted while the mixture passes through at least four heating zones, wherein a temperature of the heating zones is sequentially lowered.

9. A pharmaceutical or therapeutic supplement composition comprising a pentacyclic triterpenoid, wherein the pharmaceutical or therapeutic supplement composition comprises a solid dispersion comprising:
an amorphous form of the pentacyclic triterpenoid, and
a water-soluble polymer having a glass transition temperature lower than 170° C.

10. The pharmaceutical or therapeutic supplement composition of claim 9, wherein the pentacyclic triterpenoid is therapeutically effective in a range of from about 0.05 mg to about 20 mg/kg body weight.

11. (canceled)

12. The pharmaceutical or therapeutic supplement composition of claim 9, which is in a form of capsule, tablet, chewable tablet, soft chew, gummy, bar, oral paste, lozenge, or powder.

13. The pharmaceutical or therapeutic supplement composition of claim 9, which is free of solvent.

14. A method of treating a medical condition in a subject caused by overactive pro-inflammatory cytokines comprising administering to a subject in need of treatment for the medical condition, the pharmaceutical or therapeutic supplement composition of claim 9.

15. The method of claim 14, wherein the medical condition is kidney disease, acute kidney injury, or chronic kidney disease.

16-18. (canceled)

19. The method of claim 14, wherein the medical condition is diabetic nephropathy.

20-25. (canceled)

26. The method of claim 14, wherein the medical condition is Myasthenia gravis.

27-33. (canceled)

34. The method of claim 14, wherein the medical condition is aging.

35. The method of claim 14, wherein the medical condition is age-related sarcopenia.

36. The pharmaceutical or therapeutic supplement composition of claim 9, wherein the amorphous form of the pentacylic triterpenoid is substantially free of crystalline peaks as determined by X-ray powder diffraction.

37. The pharmaceutical or therapeutic supplement composition of claim 9, wherein the composition is stable and shows no reversal to a crystalline form after storage for at least 15 months.
